(19) United States
(12) Patent Application Publication
(10) Pub. No.: US 2004/0121452 A1
(43) Pub. Date: Jun. 24, 2004

(54) Component for biological or biochemical analysis microfluidic system

(76) Inventors: Fr?eacute;d?eacute;rick Revol-Cavalier, (US); Philippe Combette, Montpellier (FR); Alain Troussier, Echirolles (FR)

Correspondence Address: BURNS DOANE SWECKER & MATHIS L L P, POST OFFICE BOX 1404, ALEXANDRIA, VA, 22313-1404, US

(21) Appl. No.: 10/475,638

(22) PCT Filed: Feb. 20, 2003
(86) PCT No.: PCT/FR03/00569

(30) Foreign Application Priority Data
Feb. 21, 2002 (FR) 02/02204

(51) Int. Cl. C12M 1/34

(52) U.S. Cl. 435/287.2

(57) ABSTRACT

The invention concerns a component for biological or biochemical analysis microsystems formed from a support and having at least one chemically functionalised surface zone, in order to allow in said zone the formation of a chemistry for anchoring biological or biochemical elements, and/or electrically, in order to allow in said zone the formation of electrical charges. The support comprises a part (21) formed of an inert material and covered with a layer (13) of a chemically and/or electrically functionalisable material to provide said functionalised surface zone.

### TECHNICAL FIELD

The present invention concerns a component for biological or biochemical analysis microsystems. It further concerns a method for producing said component.

### STATE OF THE PRIOR ART

A microsystem for biological or biochemical analysis is produced from a support or substrate chosen so that a surface (which may comprise several zones) of said support or substrate provides one or several functions. Said function(s) may be a chemical functionally or an electrical functionality.

Chemical functionality is involved when biological or biochemical elements have to be anchored to the support. Generally, the supports are in glass or silicon, which allows the anchoring of biological or biochemical elements by a well controlled coupling chemistry, for example by silanisation.

Electrical functionality is involved for the circulation of fluids in micro-channels or micro-reservoirs. Fluid circulation microsystems generally use electrokinetic pumping, such as electro-osmosis, to make fluids circulate in the micro-channels and micro-reservoirs formed in the supports. Said pumping means require the existence of electrically active surfaces. It is the use of high electrical fields, combined with the presence of electrically active surfaces, that makes fluid flow possible. Known supports, in glass, in silica or in silicon coated with silica, are well suited to said pumping means.

Glass, silica or silicon supports coated with silica are therefore well suited to obtaining chemical and electrical functionalities.

Reference is increasingly made to the use of inert materials such as polymers, plastics and adhesives in producing said Microsystems. However, the chemistry for anchoring biological or biochemical elements on said inert materials depends on their chemical formulation and remains awkward to implement. Materials such as moulded plastics for forming micro-channels or photosensitive polymers or resins for forming microstructures would be very widely used if it were possible to easily anchor biological or biochemical elements to them. Indeed, said materials are cheap and are used in large production series.

Furthermore, electrokinetic flow is problematic in materials such as conventional polymers and requires the use of costly techniques such as plasma activation in order to generate electrically charged surfaces. However, it has been shown that this does not allow the treated surface to be activated definitively. Consequently, the system evolves over time.

### DESCRIPTION OF THE INVENTION

The present invention provides a solution to the problems described above. It allows the use of chemically inert materials (polymers, resins, plastics, adhesives, etc.) to form component supports for biological or biochemical analysis microsystems, the surfaces of said materials being treated to make them functionalisable in order to allow the anchoring of biological or biochemical elements. Said treatment consists in depositing, on the surfaces concerned, a layer of a functionalisable material. The biological or biochemical elements may then be grafted by conventional techniques, for example by a silanisation technique.

The functionalisable material may then be deposited on a flat surface of the support or on a structured surface of the support that may be in moulded plastic, in photosensitive or non-photosensitive polymer or in screen printed adhesive. The layer of functionalisable material may be deposited after the formation of a structured component. It may also be etched by the techniques used in microtechnology (plasma or chemical etching) in order to cover only a part of the surface of the component.

The deposited material may also be a material providing an electrical functionality to the component, which allows the circulation of fluids by electrokinetic pumping.

Consequently, the aim of the invention is a component for biological or biochemical analysis microsystems formed from a support and having at least one chemically functionalised surface zone, in order to allow in said zone the formation of a chemistry for anchoring biological or biochemical elements, and/or electrically, in order to allow in said zone the formation of electrical charges, characterised in that said support comprises at least one part formed of an inert material and covered with a layer of a chemically and/or electrically functionalisable material to provide said functionalised surface zone.

Preferably, said part is formed of a material chosen from among a polymer, a plastic,, a resin and an adhesive. The polymer may be a polyimide.

Said part may form the support in its entirety.

The support may comprise a substrate supporting said part. The substrate may be in a material chosen from among glass, silicon, a polymer and a metal.

Said part may be structured.

Advantageously, the functionalisable material is chosen from among silica, synthesised silica and silicon nitride.

Said surface zone may support chemical functions suited to assuring the attachment of biological elements or other chemical functions on said surface zone.

Said surface zone may support chemical functions suited to assuring the presence of electrical charges on said surface zone.

A further aim of the invention is a method for producing a component for biological or biochemical analysis microsystems from a support, said support needing to have at least one chemically functionalised surface zone to allow in said zone the formation of a chemistry for anchoring biological or biochemical elements, and/or electrically, to allow in said zone the formation of electrical charges, characterised in that it comprises the following steps:

preparing a support comprising at least one part formed of an inert material,

depositing, on said part, a layer of a chemically and/or electrically functionalisable material in order to provide said functionalised surface zone.

The preparation step may comprise the formation of said part in inert material on a substrate. Said part in inert material may be formed by deposition.

If necessary, before the step of depositing the layer of functionalisable material, said part is subjected to a structuring step.

The preparation step may comprise the moulding of said part formed of an inert material.

The method may further comprise the following steps:

before the step of depositing the layer of functionalisable material, depositing a protective layer on said part with the exception of the surface zone that is to be functionalised,

after the step of depositing the layer of functionalisable material on the whole of said part, the lift-off of the layer of functionalisable material covering the protective layer by elimination of the protective layer.

The deposition of the layer of functionalisable material may be a deposition of synthesized silica obtain by a sol-gel method.

The deposition of the layer of functionalisable material may be a deposition of silica or silicon nitride obtained by an evaporation or sputtering technique.

The method may further comprise a step of etching the layer of functionalisable material. Said etching may be a plasma etching or a chemical etching.

### BRIEF DESCRIPTION OF THE DRAWINGS

The invention will be more fully understood and other advantages and specific features will become clearer on reading the description given hereafter, given by way of indication and in nowise limitative, and by referring to the appended drawings among which:

FIGS. 1A to 1C are cross-sectional views illustrating the formation of a first component for biological or biochemical analysis Microsystems according to the invention,

FIGS. 2A to 2C are cross-sectional views illustrating the formation of a second component for biological or biochemical analysis Microsystems according to the invention.

### DETAILED DESCRIPTION OF EMBODIMENTS OF THE INVENTION

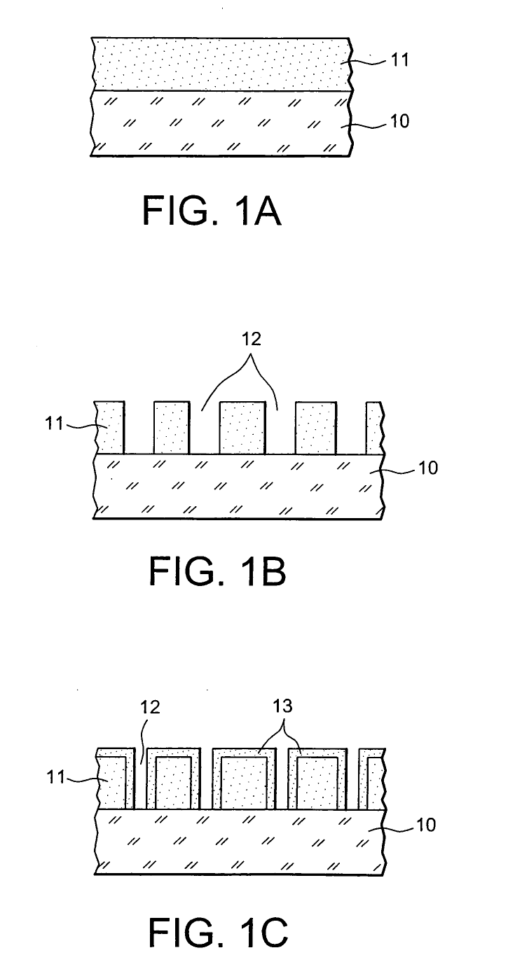

FIGS. 1A to 1C illustrate the formation of a component for biological or biochemical analysis Microsystems from a substrate in glass.

Forming biological or biochemical microsystems in a glass substrate is awkward. Beyond several micrometers of etching, the impurities contained in the glass block the etching. The glass is therefore etched by chemical means with solutions containing hydrofluoric acid. Chemical etching of glass is an isotropic etching, which limits the shape and the dimensions of the patterns that may be made. The invention makes it possible to do away with the etching of the glass by forming the component in a structurable material such as a photosensitive or non-photosensitive polymer, a photosensitive resin or an adhesive. Said structurable material is then covered with a functionalisable material such as a synthetic silica, a silica or a silicon nitride obtained by evaporation or by sputtering, or any other functionalisable material that one wishes to deposit on the structurable material.

The functionalisation is no longer carried out directly on the glass substrate but by the intermediary of the layer of functionalisable material deposited on the structurable material. The substrate supporting the structurable material may then be of diverse nature. It may be in glass, in silicon, in polymer, in metal or in any other material on which one can deposit a layer of structurable material.

FIG. 1A shows a substrate 10, formed of a sheet of silicon of 100 mm diameter. On the substrate 10, a layer of structurable material 11 is deposited. It is a photosensitive polyimide polymer commercialised under the trade name “Probimide 7510”. The layer 11 is deposited with a spin coater, at a speed of 3000 rpm then annealed at 110° C. on a heating plate. The substrate 10 and the layer 11 form a support.

Layer 11 is radiated with ultra violet rays through a mask, then developed in order to obtain the desired component. This is what is shown in FIG. 1B. Layer 11 is structured by the presence of openings 12 exposing the substrate 10.

The polyimide is then annealed at 150° C. on a heating plate then at 300° C. in a thermal treatment oven.

FIG. 1C shows the component obtained after the deposition of a layer of functionalisable material 13 on the layer 11. Said functionalisable material is for example the synthetic silica commercialised under the trade name “SOG 512” by the Honeywell company. The synthetic silica is deposited with a spin coater at 1000 rpm then annealed on a heating plate first at 110° C. then at 250° C.

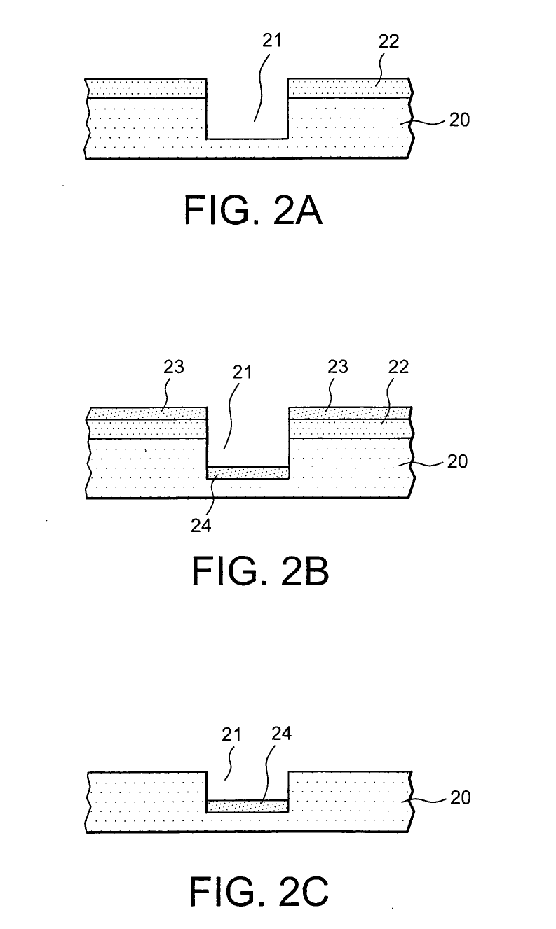

FIGS. 2A to 2C illustrate the formation of a component from a moulded plastic having a functionalisable micro-channel.

The use of moulded plastic for forming biological or biochemical components is a very promising technique since the component may be functionalised. The invention allows the functionalisation, by a synthetic silica, of a part of the moulded structure, for example a micro-channel.

FIG. 2A shows a support 20 in moulded plastic, provided with a channel 21 formed by moulding. On the upper part of the support 20, a layer 22 of protective material is deposited. This may be a photosensitive resin used in microtechnology. Said resin is deposited with a spin coater at 2000 rpm then annealed at 90° C. in an oven.

Then, as shown in FIG. 2B, a layer of silica, obtained by evaporation of a load of silica in an evaporation unit, is deposited on the support. The layer of silica forms a deposit 23 on the layer 22 of protective material and a deposit 24 on the base of the channel 21.

The protective layer is then lifted off in a chemical bath, for example in an acetone bath. Its dissolution leads to the lift off of the silica deposited on its surface. The result obtained is shown in FIG. 2C. Only the layer of silica 24 contained at the base of the channel 21 remains.

The invention finds an application in all cases where one wishes to biologically or biochemically functionalise a part or the whole of a surface of a material chemically inert to the functionalisation. It finds a further application when it is necessary to use a layer of electrically active material.

Depending on the nature of the functionalisable material, different techniques may be used to functionalise it. In the case of materials such as silicon, silicon oxide, silicon nitride or synthetic silica, a silanisation treatment makes it possible to attach to the surface of said materials chemical functions that will subsequently assure the attachment of biological elements or chemical functions.

Different types of silane may be used. Each has its own protocol for attaching to the surface of the material to be functionalised. The choice of silane to use depends on the chemical function that one wishes to use either directly, or for the subsequent carrying out of a chemical reaction or the attachment of a biological element. Among the most widely used silanes, one may cite aminopropyl triethoxysilane, aminopropyl dimethylethoxy silane, epoxy silane, 2-(hydroxyethyl)-3-aminopropyl triethoxysilane.

By way of example, the silanisation protocol used for aminopropyl triethoxysilane is as follows:

treating the surface concerned by an oxygen plasma (Nextral 310) at 150 watts for 30 seconds to create silanol functions on the surface;

incubating in a 10% silane solution in 95% ethanol for 12 hours;

rinsing in distilled water;

rinsing in 95% ethanol;

annealing at 110° C. for 3 hours in an oven.

One may directly attach synthesized oligonucleotides with an aldehyde function or by the intermediary of a glutaraldehyde if the oligonucleotides are synthesized with an NH₂ function.

This silanisation technique makes it possible to attach oligonucleotides, proteins or any biological or chemical element compatible with the functions present on the silane attached to the functionalised material (amine, aldehyde acid, activated ester functions, etc.).

If the material to be functionalised is a layer of gold, one uses the attachment of thiols or disulphide compounds on the surface of said metallic layer. As for silanes, different thiols make it possible to obtain on the surface of the layer to be functionalised the chemical functions necessary for the desired chemical reactions. The techniques for attaching thiols on a metallic surface are known, for example through the following document “Formation of Monolayer Films by the Spontaneous Assembly of Organic Thiols from Solution onto Gold” by C. D. BAIN et al., J. Am. Chem. Soc., 1989, Vol. III, N^{o} 1, pages 321 to 335.

Again by way of example, one may cite the grafting of mercapto-propionic acid or cystamin by incubating a 1 mM solution for 3 hours in absolute ethanol at ambient temperature.

For an electrical functionalisation, one may obtain electrical charges on the surface of synthetic silica, silicon, silicon nitride and silicon oxide by grafting an aminopropyl triethoxysilane on the layer to be functionalised according to the protocol described here-above. A treatment in acid medium (for example 0.2 M HCl) makes it possible to protect the amine group of the silane and obtain electrical charges on the surface of the functionalised material.

The invention may advantageously be used in the field of biological and biochemical analysis systems as well as in “lab-on-chip” systems.

1. Component for biological or biochemical analysis Microsystems formed from a support and having at least one chemically functionalised surface zone, in order to allow in said zone the formation of a chemistry for anchoring biological or biochemical elements, and/or electrically, in order to allow in said zone the formation of electrical charges, characterised in that said support comprises at least one part (11, 20) formed of an inert material and covered with a layer (13, 24) of a chemically and/or electrically functionalisable material to provide said functionalised surface zone.

2. Component according to claim 1, characterised in that said part (11, 20) is formed of a material chosen from among a polymer, a plastic, a resin and an adhesive.

3. Component according to claim 2, characterised in that the polymer is a polyimide.

4. Component according to claim 1, characterised in that said part (20) forms the support in its entirety.

5. Component according to claim 1, characterised in that the support comprises a substrate (10) supporting said part (11).

6. Component according to claim 5, characterised in that the substrate (10) is in a material chosen from among glass, silicon, a polymer and a metal.

7. Component according to any of claims 1 to 6, characterised in that said part (11, 20) is structured.

8. Component according to any of claims 1 to 7, characterised in that the functionalisable material is chosen from among silica, synthesised silica and silicon nitride.

9. Component according to any of claims 1 to 8, characterised in that said surface zone supports chemical functions suited to assuring the attachment of biological elements or other chemical functions on said surface zone.

10. Component according to any of claims 1 to 8, characterised in that said surface zone supports chemical functions suited to assuring the presence of electrical charges on said surface zone.

11. Method for producing a component for biological or biochemical analysis Microsystems from a support, said support needing to have at least one chemically functionalised surface zone to allow in said zone the formation of a chemistry for anchoring biological or biochemical elements, and/or electrically, to allow in said zone the formation of electrical charges, characterised in that it comprises the following steps:
preparing a support comprising at least one part (11, 20) formed of an inert material,
depositing, on said part (11, 20), a layer (13, 24) of a chemically and/or electrically functionalisable material in order to provide said functionalised surface zone.

12. Method according to claim 11, characterised in that the preparation step comprises the formation of said part (11) in inert material on a substrate (10).

13. Method according to claim 12, characterised in that said part (11) in inert material is formed by deposition.

14. Method according to any of claims 11 to 13, characterised in that, before the step of depositing the layer of functionalisable material, said part (11) is subjected to a structuring step.

15. Method according to claim 11, characterised in that the preparation step comprises the moulding of said part (20) formed of an inert material.

16. Method according to any of claims 11 to 15, characterised in that it further comprises the following steps:
before the step of depositing the layer of functionalisable material, depositing a protective layer (22) on said part (20) with the exception of the surface zone that is to be functionalised,
after the step of depositing the layer of functionalisable material (23, 24) on the whole of said part (20), the lift-off of the layer of functionalisable material (23) covering the protective layer (22) by elimination of the protective layer.

17. Method according to any of claims 11 to 16, characterised in that the deposition of the layer of functionalisable material (13) is a deposition of synthesized silica obtained by a sol-gel method.

18. Method according to any of claims 11 to 16, characterised in that the deposition of the layer of functionalisable material is a deposition (23, 24) of silica or silicon nitride obtained by an evaporation or sputtering technique.

19. Method according to any of claims 11 to 18, characterised in that it further comprises a step of etching the layer of functionalisable material.

20. Method according to claim 19, characterised in that the etching of the layer of functionalisable material is a plasma etching or a chemical etching.
